(19) United States
(12) Patent Application Publication
(10) Pub. No.: US 2024/0108860 A1
(43) Pub. Date: Apr. 4, 2024

(54) LOCKING CATHETER AND METHODS OF USING SAME

(71) Applicant: Uresil, LLC, Skokie, IL (US)

(72) Inventors: Tyler Panian, Naperville, IL (US); Scott Irving, Chicago, IL (US)

(21) Appl. No.: 18/376,723

(22) Filed: Oct. 4, 2023

Related U.S. Application Data
(60) Provisional application No. 63/413,155, filed on Oct. 4, 2022.

(51) Int. Cl. A61M 25/01; A61M 27/00

(52) U.S. Cl. CPC A61M 25/0136; A61M 27/00

(57) ABSTRACT

Provided herein is a catheter lock. The catheter lock includes a hub having a first axial extension, a second axial extension, and an intersecting opening with at least one hub feature formed on an inner surface thereof; a button having an outer surface mirroring the inner surface of the intersecting opening and at least one button feature formed thereon, an elongated transverse opening, a cutout section extending along the outer surface of the button, and a hole in a top portion thereof; and a locking article configured to extend through the first axial extension, along the cutout section, and through the hole. The button is arranged and disposed to slide within the intersecting opening of the hub, and the at least one button feature is arranged and disposed to engage one of the at least one hub features. Also provided herein are an axial catheter lock and a locking catheter.

### CROSS-REFERENCE TO RELATED APPLICATION(S)

The present application claims priority under 35 U.S.C. § 119(e) to U.S. Provisional Patent Application No. 63/413,155, filed Oct. 4, 2022, which application is incorporated herein by reference in its entirety.

### BACKGROUND OF THE INVENTION

Various fluids, such as blood, serum, bile, or pus, often accumulate in a patient's body as a result of injury or disease. If accumulation of these fluids persists, it can lead to rupturing, infection of surrounding tissue, and/or other complications. In view thereof, drainage catheters have been developed to remove these fluids from a patient.

Drainage catheters typically include an elongated tube with a proximal end and a distal end. The distal end is generally flexible and includes a plurality of drainage holes. During use, the distal end of the drainage catheter is inserted into the patient while in a straight configuration, then manipulated into a desired drainage configuration once positioned in the area of fluid accumulation. This manipulation of the distal end is usually accomplished through a string, suture, or other tensioning member that extends through the elongated tube. For example, once the distal end is positioned in the area of fluid accumulation, a user may pull on the string from the proximal end, which causes the distal end to bend, curve, flex, or otherwise change configuration. Once the desired configuration has been reached, the tensioning member is held by the user and/or wrapped around the proximal end of the elongated tube, permitting the fluid to enter the drainage holes and flow out of the patient through the drainage catheter.

Similar to manually holding the tensioning member throughout draining, other existing articles and methods for securing the tensioning member can be insecure. Additionally, it can be difficult to reposition or further manipulate the drainage catheter after securing the tensioning member with existing articles and methods. Furthermore, existing articles and methods can make it difficult to release the tensioning member and return the distal end to a straight configuration for removal from the patient.

Accordingly, there is a need in the art for articles and methods that improve on existing drainage catheters by providing mechanisms for securely holding and releasing the drainage configuration of the distal end. The present invention addresses this need.

### SUMMARY

In one aspect provided herein is a catheter lock including a hub, a button, and a locking article. In some embodiments, the hub includes a first axial extension, a second axial extension, and an intersecting opening with at least one hub feature formed on an inner surface thereof. In some embodiments, the button includes an outer surface mirroring the inner surface of the intersecting opening, at least one button feature formed on the outer surface, an elongated transverse opening, a cutout section extending along the outer surface of the button, and a hole in a top portion thereof. In some embodiments, the locking article is configured to extend through the first axial extension, along the cutout section, and through the hole. In some embodiments, the button is arranged and disposed to slide within the intersecting opening of the hub. In some embodiments, the at least one button feature is arranged and disposed to engage one of the at least one hub features.

In some embodiments, the at least one hub feature includes a first hub feature and a second hub feature. In some embodiments, the at least one button feature includes two sets of button features, each set including a first button feature, a second button feature, and a third button feature. In some embodiments, the first button feature of the first set engages the first hub feature and the first button feature of the second set engages the second hub feature to hold the button in a first position within the hub. In some embodiments, the second button feature of the first set engages the first hub feature and the second button feature of the second set engages the second hub feature to hold the button in a second position within the hub. In some embodiments, the third button feature of the first set engages the first hub feature and the third button feature of the second set engages the second hub feature to hold the button in a third position within the hub.

In some embodiments, the cutout section extends from a first side of the elongated transverse opening to a second side of the elongated transverse opening. In some embodiments, the third button feature of the first set of button features is positioned within the cutout section. In some embodiments, the button forms a fluid-tight seal with the hub. In some embodiments, the elongated transverse opening aligns with the first axial extension and the second axial extension when the button is positioned within the hub.

In another aspect, provided herein is a catheter lock including a hub, a lock member, and a locking article. In some embodiments, the hub includes a first axial extension, a second axial extension with at least one hub feature formed on an inner surface thereof, and a hole formed in a surface thereof. In some embodiments, the lock member includes an outer surface mirroring the inner surface of the second axial extension, at least one lock member feature formed on the outer surface, an axial opening extending through the lock member, and at least one surface element on the outer surface of the lock member. In some embodiments, the at least one surface element is configured to contact an inner surface of the hub only when the lock member is in a closed position. In some embodiments, the locking article configured to extend through the first axial extension, between the outer surface of the lock member and the inner surface of the hub, and through the hole. In some embodiments, the lock member is arranged and disposed to slide within the second axial extension of the hub. In some embodiments, the at least one lock member feature is arranged and disposed to engage one of the at least one hub features to maintain a position of the lock member within the second axial extension. In some embodiments, the axial opening extending through the lock member is arranged and disposed to align with the first axial extension when the lock member is positioned within the second axial extension.

In some embodiments, the catheter lock further includes a grommet arranged and disposed to be positioned within the hole formed in the hub, the grommet forming a liquid tight seal with the locking article. In some embodiments, the at least one lock member feature and the at least one hub feature are arranged and disposed to maintain the lock member in an open position or the closed position. In some embodiments, the at least one surface element is arranged and disposed to pin the locking article between the outer surface of the lock member and the inner surface of the hub when the lock member is in the closed position.

In a further aspect, provided herein is a locking catheter including the catheter lock according to any of the embodiments disclosed herein, and an elongated tube member having a proximal end and a distal end. In some embodiments, the proximal end of the elongated tube member is coupled to the first axial extension of the catheter lock. In some embodiments, the distal end of the elongated tube member is flexible and includes at least one opening formed therein. In some embodiments, the locking article is arranged and disposed to be coupled to the distal end of the elongated tube member before extending through the first axial extension and through the hole.

In some embodiments, the distal end of the elongated tube member includes a first curved or looped configuration. In some embodiments, the locking catheter further includes a removable stylet, the stylet being arranged and disposed to straighten the distal end when inserted into the elongated tube member. In some embodiments, pulling a free end of the locking article extending through the hole applies tension to the locking article. In some embodiments, the tension holds a desired position of the distal end. In some embodiments, when in a closed position the catheter lock maintains the tension in the locking article to secure a position of the distal end.

### BRIEF DESCRIPTION OF THE DRAWINGS

FIGS. 1A-B show images of a locking catheter. (A) Locking catheter. (B) Stylet.

FIGS. 2A-C show images of a locking catheter. (A) Perspective view of a locking catheter. (B) Side view of a locking catheter. (C) Top view of a locking catheter.

FIG. 3 shows an exploded view of a catheter lock.

FIGS. 4A-B show images of a catheter lock in an open position.

FIGS. 5A-B show images of a catheter lock in a closed position.

FIGS. 6A-C show images illustrating how the catheter lock secures the suture. (A) Catheter lock in open position with suture extending therefrom. (B) Catheter lock in closed position with suture extending therefrom. (C) Front section view of catheter lock showing where suture is held therein.

FIGS. 7A-D show images illustrating the path of a suture through the locking catheter. (A) Catheter lock button inserted in catheter lock to first stop with suture tied to button and extending into catheter tubing. (B) Suture returning from catheter tubing after looping through a distal end, wrapping around the catheter lock button, passing through an opening in the catheter lock button, and then through a grommet. (C) Catheter lock button moved from first stop position to an unlocked position in the catheter lock. (D) Grommet inserted into catheter lock button and catheter tubing seated within catheter lock.

FIGS. 8A-C show images illustrating an axial catheter lock according to an alternate embodiment of the disclosure. (A) Exploded view of an axial catheter lock. (B) Side view of an axial catheter lock in the unlocked position. (C) Side view of an axial catheter lock in the locked position.

FIGS. 9A-D show images illustrating how the axial catheter lock secures the suture. (A) Perspective view of the axial catheter lock with the suture in an unlocked position. (B) Side view of the axial catheter lock with the suture in an unlocked position. (C) Perspective view of the axial catheter lock with the suture in a locked position. (D) Side view of the axial catheter lock with the suture in a locked position.

FIGS. 10A-C show images illustrating assembly of the axial catheter lock. (A) Exploded view showing tying of the suture to the axial lock and feeding of the suture through the catheter. (B) Exploded view showing return of the suture from the catheter and feeding of the suture through the axial housing and the grommet. (C) Perspective view of the assembled axial catheter lock in an unlocked position.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

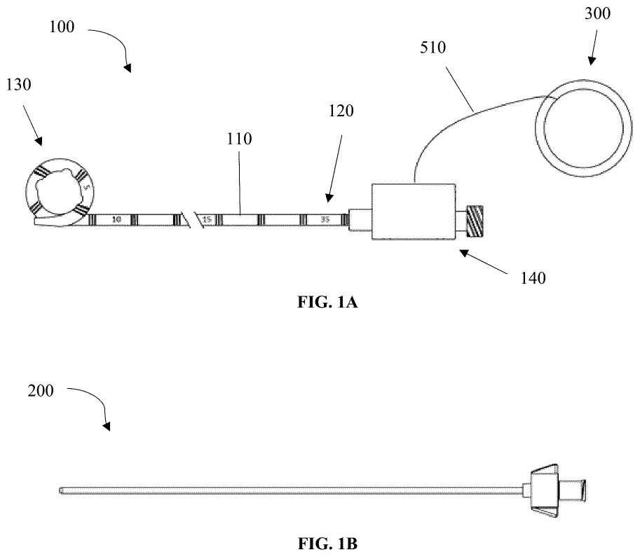

Provided herein in some embodiments, is a locking catheter 100 (FIGS. 1A-B). The locking catheter 100 includes an elongated tube member 110 having a proximal end 120 and an opposite distal end 130. The distal end 130 includes one or more openings (e.g., apertures, holes, bores, etc.) extending between an interior and an exterior of the elongated tube member 110. In some embodiments, the distal end 130 is also flexible, such that when a force is applied the distal end 130 will take on a different configuration, and when the force is removed the distal end will return or substantially return to the original configuration. For example, in one embodiment, the distal end 130 of the elongated tube 110 is curved or looped and includes a plurality of holes. In such embodiments, the locking catheter 100 includes a removable stylet 200 (FIG. 1B) configured for insertion within the elongated tube member 110. When inserted, the removable stylet 200 straightens the distal end 130 of the elongated tube 110. After positioning of the locking catheter 100, the stylet 200 is removed from the elongated tube 110 and the distal end 130 returns or substantially returns to the original configuration (e.g., curved or looped configuration).

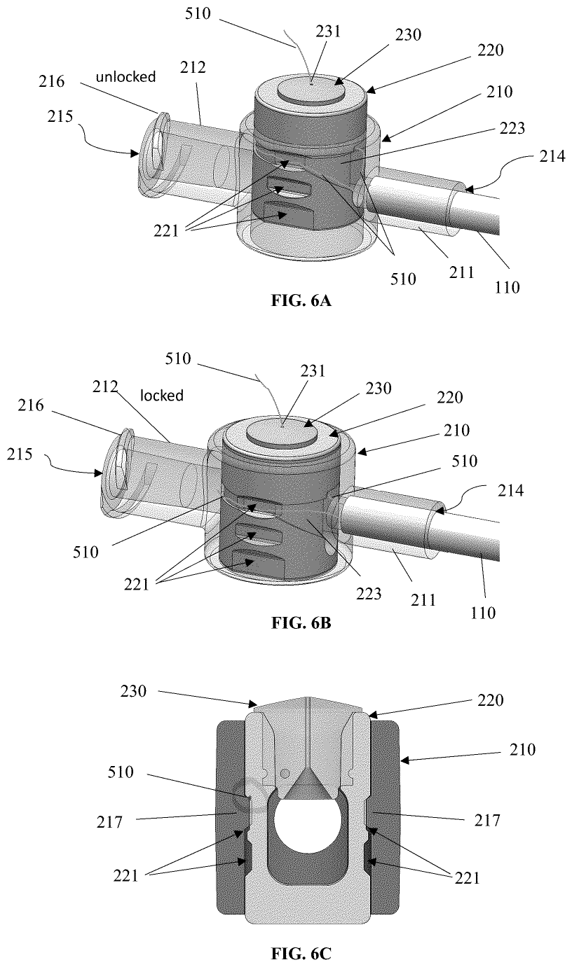

When the stylet 200 is removed/not inserted (e.g., after positioning of the locking catheter), the distal end 130 may be secured in a desired configuration through a locking article 510 (FIG. 6). In some embodiments, the locking article 510 extends through the proximal end 120 and/or a portion of the elongated tube 110, and is secured to the distal end 130. Suitable locking articles 510 include, but are not limited to, thread, wire, suture, or any other article capable of extending through at least a portion of the elongated tube 110 and being coupled to the distal end 130. For example, in some embodiments, the locking article 510 includes a suture extending through the proximal end 120, within a portion of the elongated tube 110, and being secured to the distal end 130. In such embodiments, the locking article 510 is configured such that when pulled or held tight it secures, or “locks,” the distal end 130 in a desired configuration relative to a more proximal portion of the elongated tube 110. When secured, the distal end 130 is able to withstand force on the locking catheter 100 in order to maintain the desired and/or original configuration. Prior to removal of the locking catheter 100, the locking article 510 is released, permitting the distal end 130 to be manipulated (e.g., straightened) for removal.

Additionally or alternatively, in some embodiments, the locking article 510 may be used to modify the position of the distal end 130 relative to a more proximal portion of the elongated tube 110 (e.g., increase the curvature, tighten the loop, pull the distal end closer to a more proximal portion). For example, in some embodiments, when the locking article 510 is pulled tight it applies a force to the distal end 130, pulling the distal end 130 adjacent to an opening in the elongated tube 110 through which the locking article 510 exits prior to being secured to the distal end 130. As will be appreciated by those skilled in the art, the location of opening through which the locking article 510 exits the elongated tube 110 prior to being coupled to the distal end 130 and/or the amount of force applied to the locking article 510 may be adjusted to provide a desired configuration of the distal end 130 in the locked position. Although described herein primarily with respect to a suture and a curved configuration, as will be appreciated by those skilled in the art, the disclosure is not so limited and may include any other suitable article 510 for securing the distal end 130 and any other suitable desired configuration (e.g., looped, twisted, bent).

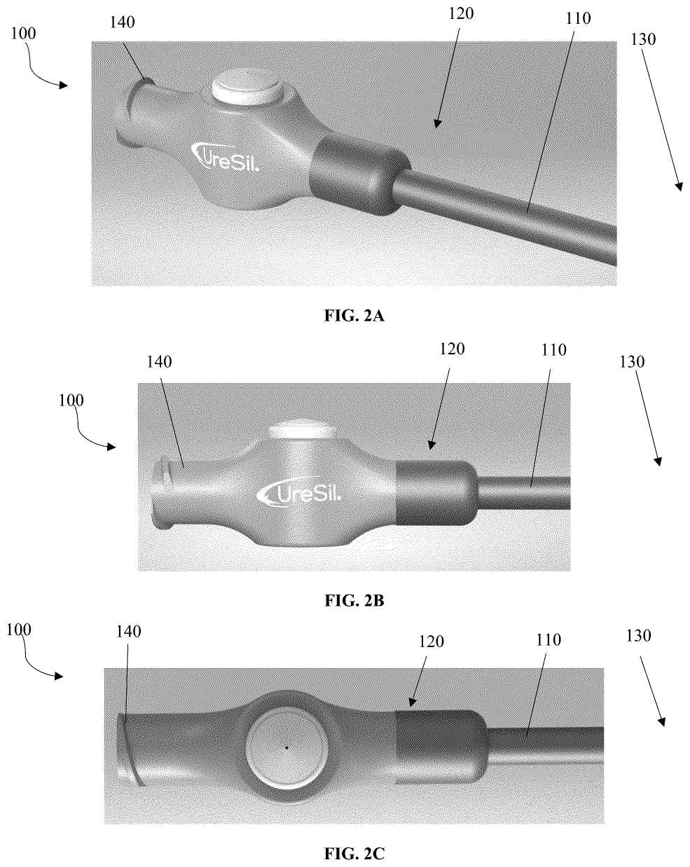

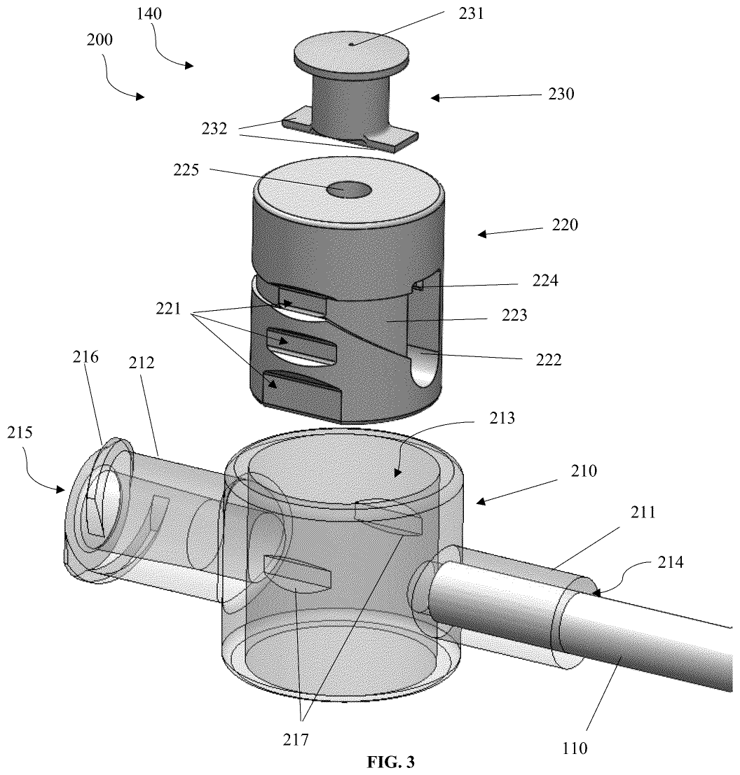

Opposite the distal end 130 with respect to the elongated tube member 110, the proximal end 120 includes a locking device 140 (FIGS. 2A-C), which is also referred to herein as a catheter lock. The catheter lock 140 is arranged and disposed to secure a position of the locking article 510. In some embodiments, as illustrated in FIGS. 3-7C, the catheter lock 140 includes a button lock arrangement 200 with a hub 210, a button 220, and a grommet 230.

Referring to FIG. 3, the hub 210 includes a first axial extension 211, a second axial extension 212, and an intersecting opening 213. A first axial opening 214 in the first axial extension 211 is arranged and disposed to receive the elongated tube member 110 therein, while a second axial opening 215 in the second axial extension 212 is arranged and disposed to receive and communicate fluid to (e.g., flushing) or from (e.g., draining) the first axial extension 211. In some embodiments, the second axial extension 212 includes threading 216 arranged and disposed for coupling with a mating member. The intersecting opening 213 extends through at least a portion of the hub 210 and intersects a flow path between the first axial extension 211 and the second axial extension 212. Although shown primarily as being perpendicular or substantially perpendicular to the first axial extension 211 and the second axial extension 212, the intersecting opening 213 may be at any angle with respect thereto.

Still referring to FIG. 3, the intersecting opening 213 is arranged and disposed to receive the button 220 therein. The button 220 includes an outer size and shape configured to movably fit within the intersecting opening 213 of the hub 210. In some embodiments, the button 220 includes a size and shape that mirrors the intersecting opening 213, such that the button 220 can slide within the intersecting opening 213 while maintaining contact between the outer surface of the button 220 and the inner surface of the intersecting opening 213. Although shown herein primarily with respect to a circular or cylindrical shape, the button 220 and intersecting opening 213 are not so limited and may include any other suitable shapes, such as, but not limited to, square, rectangular, oval, or hexagonal. Additionally or alternatively, in some embodiments, the button 220 and the hub 210 are formed from materials that, in combination with the sizes and shapes thereof, create a fluid tight seal therebetween. For example, the button 220 may include a soft thermoplastic while the hub 210 includes a hard thermoplastic, or vice versa, which together provide a watertight seal. Suitable soft thermoplastics include, but are not limited to, LDPE, HDPE, PP, acetal, or any other suitable soft plastic. Suitable hard plastics include, but are not limited to, ABS, PC, or any other suitable hard plastic.

In some embodiments, the intersecting opening 213 includes one or more hub features 217 configured to engage a corresponding button feature 221 on the button 220. For example, in one embodiment, the one or more hub features 217 include rounded or angled projections configured to engage with the corresponding button feature(s) 221. When positioned within the intersecting opening 213, each of the button features 221 is configured to engage one of the hub features 217 at a specific position of the button 220. For example, in some embodiments, as illustrated in FIG. 3, the button 220 includes three button features 221 on opposite sides of the button 220. In one such embodiment, the bottom button feature 221 is configured to engage the hub feature 217 in order to hold the button 220 in a first position. In another embodiment, when pressure is applied to the button 220, the bottom button feature 221 will slide past the hub feature 217 and the middle button feature 221 will engage with the hub feature 217 to hold the button 220 in a second position. In a further embodiment, when additional pressure is applied to the button 220, the middle button feature 221 will slide past the hub feature 217 and the top button feature 221 will engage with the hub feature 217 to hold the button in a third position.

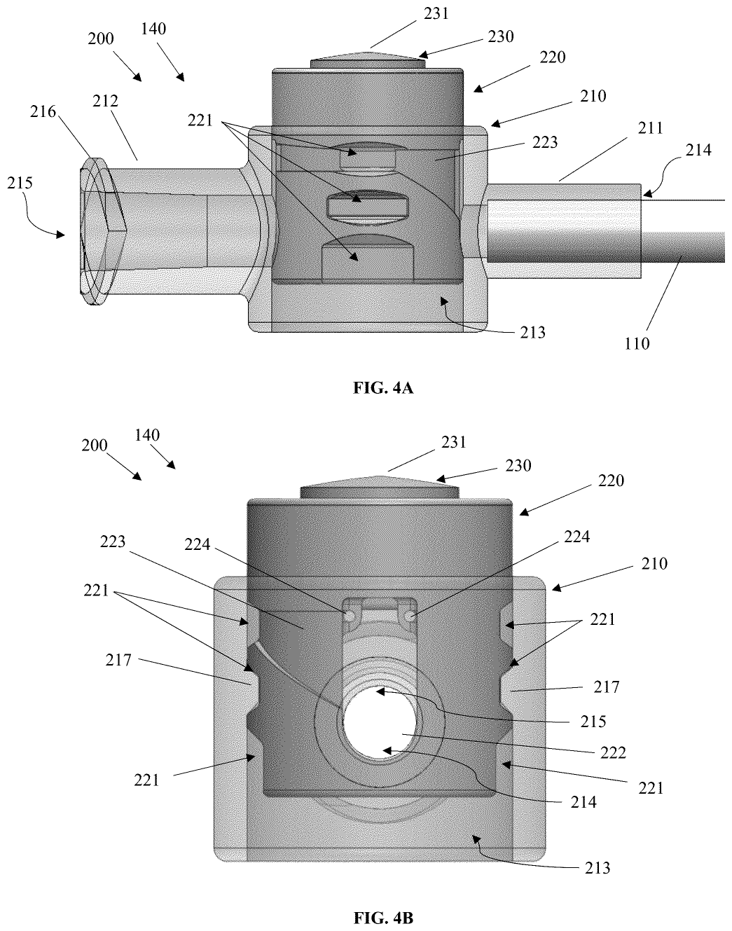

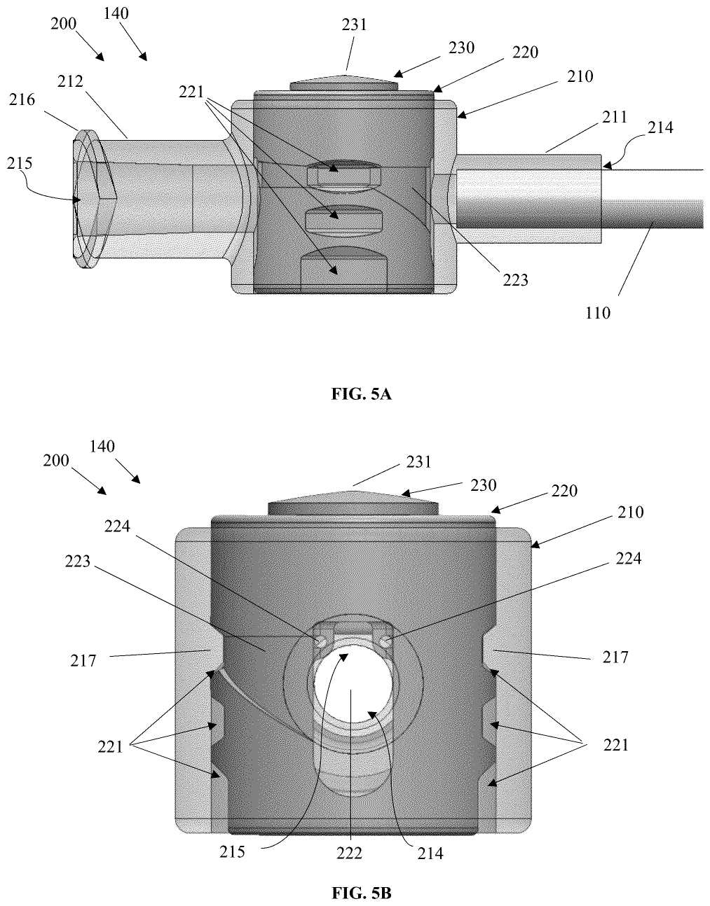

In certain positions, an elongated transverse opening 222 extending through the button 220 is aligned with the first axial opening 214 and the second axial opening 215. For example, referring to FIGS. 4A-B, when the button 220 is in an open position (e.g., the second position when the button 220 has three button features 221), a bottom section of the elongated transverse opening 222 is aligned with first axial opening 214 and the second axial opening 215. Turning to FIGS. 5A-B, when the button 220 is in a closed position (e.g., the third position when the button 220 has three button features 221), a top section of the elongated transverse opening 222 is aligned with first axial opening 214 and the second axial opening 215. As such, due to the shape of the elongated transverse opening 222, the button 220 is able to maintain fluid communication between the first axial opening 214 and the second axial opening 215 when in both the open and closed positions. Although described herein with respect to the open position corresponding to the second position and the closed position corresponding to the third position, as will be appreciated by those skilled in the art, the disclosure is not so limited and may vary depending upon the number of button features 221.

Referring again to FIG. 3, the button 220 further includes a cutout section 223 that extends along an outer surface of the button 220, from one side of the elongated transverse opening 222 to the other side of the elongated transverse opening 222. The cutout section 223 includes any suitable shape that passes over the button feature 221 corresponding to a closed position, and may extend along either or both sides of the button 220. As illustrated in FIG. 6A, when the button 220 is in the open position, the locking article 510 passes freely along the cutout section 223, between the outside surface of the button 220 and the inside surface of the hub 210. In such embodiments, a free end of the locking article 510 extending from the catheter lock 140 may be pulled to tighten the locking article 510 between the distal end 130 of the elongated tube 110 and the free end of the locking article 510 (e.g., secure a position of the distal end 130). Similarly, when a pulling force is removed from the locking article 510 while the button 220 is in the open position, the distal end 130 may be extended or moved (e.g., extended into a straight position for insertion or removal). However, as shown in FIGS. 6B-C, when the button 220 is in the closed position, the locking article 510 is pinned between the hub feature 217 and the corresponding button feature 221, preventing movement of the locking article 510 within the cutout section 223. As such, closing the button 220 when the locking article 510 is pulled tight secures a position of the locking article 510, preventing release of any tension on the locking article 510 when the pulling force is removed from the free end thereof. By maintaining the position of the locking article 510, the closing of the button 220 secures, or “locks,” a position of the distal end 130 while permitting the pulling force to be released from the free end of the locking article 510.

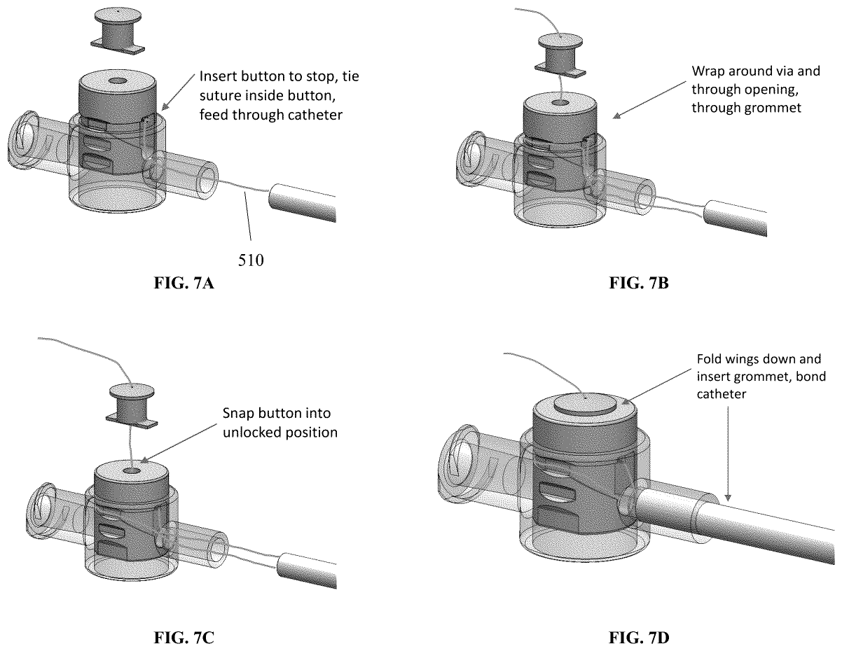

As will be appreciated by those skilled in the art, the locking article 510 may include any suitable configuration for securing the desired configuration of the distal end 130. For example, in some embodiments, as illustrated in FIGS. 7A-D, the article 510 may be tied to a fixation point 224 (FIGS. 3 and 7A), fed into the proximal end 120, through a portion of the elongated tube 110 (FIG. 7A), and out through an opening in the elongated tube 110 near the distal end 130; looped through the distal end 130; fed back into the opening and through the elongated tube 110 (FIGS. 7B-C); passed along the cutout section 223; passed into the elongated transverse opening 222; and fed through a hole 225 in the top of the button 220 (FIGS. 7B-D). A free end of the locking article 510 extending through the hole 225 may optionally be coupled to a pull-ring, bar, or any other element 300 for helping to grip the locking article 510 when pulling tight. In such embodiments, when the portion of the locking article 510 extending through the hole 225 in the top of the button 220 is pulled a tension is applied to the distal end 130, securing the position of the distal end 130 relative to the rest of the elongated tube 110. Alternatively, the locking article 510 may be fixed directly to the distal end 130 (instead of the fixation point 223), fed through the elongated tube 110, and along the button 220 as otherwise discussed above. Additionally or alternatively, the locking article 510 may be wrapped around the button 220 before or after being fed through the elongated tube 110.

Turning again to FIG. 3, in some embodiments, the catheter lock 200 includes a grommet 230. In some embodiments, the grommet 230 is configured to be inserted into the hole 225 in the top of the button 220. In such embodiments, the free end of the locking article 510 is passed through a grommet hole 231 in the top of the grommet 230 after existing the button 220. The grommet hole 231 provides a leak-tight seal against the locking article 510, while the grommet 230 provides a leak tight seal against the hole 225 in the top of the button 220, such that any fluid entering the elongated transverse opening 222 from the first axial opening 214 passes through the button 220 and into the second axial opening 215 without leaking. Additionally or alternatively, in some embodiments, the grommet 230 includes a securing feature, such as tabs 232 extending laterally therefrom. The tabs 232 are arranged and disposed to fold during insertion of the grommet 230 and then unfold to secure the position of the grommet 230 within the button 220. Other securing features include, but are not limited to, mating threads, corresponding recesses and projections, or any other suitable feature for engaging the button 220. Although shown herein primarily with respect to a separate grommet and button, as will be appreciated by those skilled in the art, the disclosure is not so limited and may include any other suitable arrangement for creating a leak-tight seal with the article. For example, the grommet 230 may be permanently fixed to the button 220, or the hole 225 in the button 220 may create a leak-tight seal with the locking article 510 directly without a grommet.

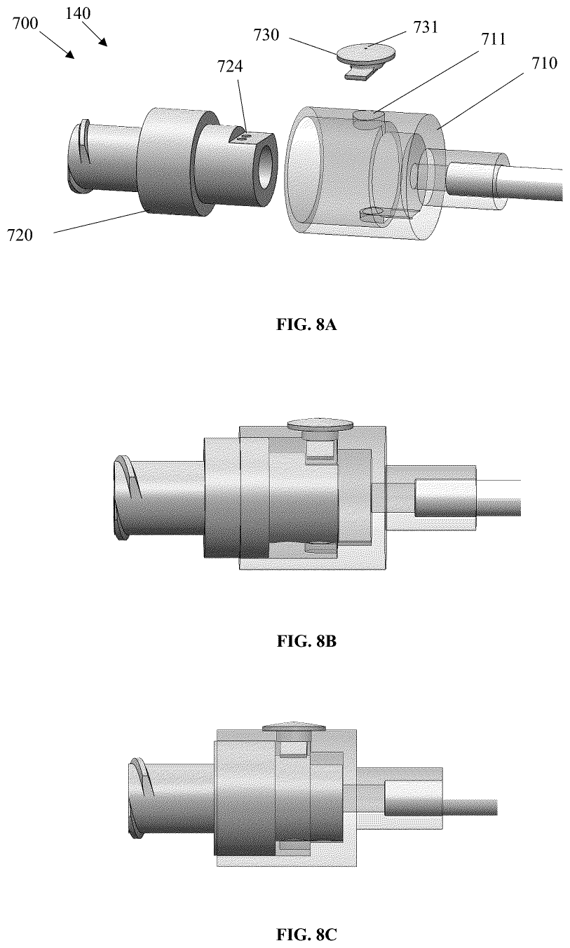

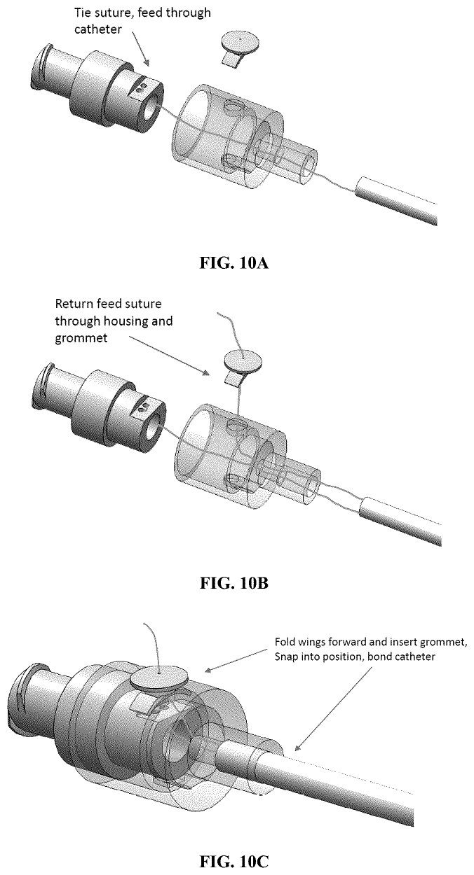

In an alternate embodiment, as illustrated in FIGS. 8A-10C, the catheter lock 140 includes an axial lock arrangement 700 with an axial hub 710, an axial lock member 720, and a grommet 730. In contrast to the button lock arrangement 200, where the button 220 is inserted into the intersecting opening 213, the axial lock member 720 of the axial lock arrangement 700 is coaxial or substantially coaxial with the elongated tube 110. As shown in FIGS. 8A-10C, the axial lock member 720 includes an axial opening extending therethrough, and replaces the second axial extension 212 of the button lock arrangement 200. In such embodiments, the axial lock member 720 is positioned within the axial hub 710, and may be moved axially between the unlocked (FIG. 8B) and locked (FIG. 8C) positions. For example, in some embodiments, as illustrated in FIGS. 8A-10C, the axial lock member 720 is slid towards the first axial extension 211 to move the axial lock arrangement 700 from the unlocked position (FIG. 8B) to the locked position (FIG. 8C), or vice versa.

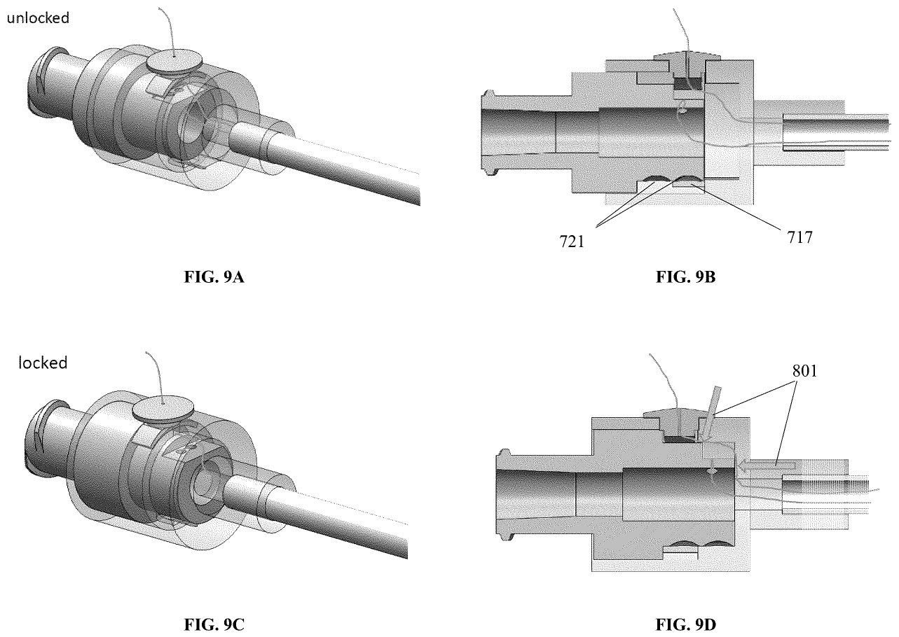

Similar to the button lock arrangement 200, in some embodiments, the axial lock arrangement 700 includes a fixation point 724 on the axial lock member 720. One end of the locking article 510 is secured to the fixation point 724, while the other end of the locking article 510 is fed through a grommet hole 731 in the grommet 730 after being looped through the elongated tube 110 and secured to the distal end 130. Once inserted into a perpendicular opening 711 in the axial hub 710, the grommet 730 provides a leak-tight seal against the axial hub 710, while the grommet hole 731 provides a leak-tight seal against the locking article 510. Referring to FIGS. 9A-B, when the axial lock member 720 is in an unlocked position, the distal end 130 of the elongated tube member 110 may be moved/held in a desired position by pulling the free end the locking article 510 extending through the grommet hole 731. Turning to FIGS. 9C-D, moving the axial lock member 720 to a locked position pins the locking article 510 between an outer surface of the axial lock member 720 and an inner surface of the axial hub 710 at one or more contact points 801 (FIG. 9D), preventing movement of the locking article 510 through the axial hub 710. By preventing movement of the locking article 510 through the axial hub 710, the axial lock member 710 maintains any tension applied to the locking article 510 before being moved to the locked position, and thus secures, or “locks,” the position of the distal end 130.

Still referring to FIGS. 9A-D, in some embodiments, the axial lock member 720 includes one or more axial lock features 721 arranged and disposed to engage with one or more axial hub features 717 to maintain a position of the axial lock member 720 within the axial hub 710. For example, in some embodiments, the one or more axial lock features 721 include a first recessed portion and a second recessed portion, while the one or more axial hub features 717 include a projection. In one embodiment, as illustrated in FIG. 9B, the first recessed portion engages with the projection to maintain the axial lock member 720 in an unlocked position within the axial hub 710. In another embodiments, as illustrated in FIG. 9D, the second recessed portion engages with the projection to maintain the axial lock member 720 in a locked position within the axial hub 710. Although discussed herein primarily with respect to recessed portions and projections, as will be appreciated by those skilled in the art, the axial lock features and axial hub features are not so limited and may include any other suitable shape or configuration. For example, the axial lock features may include projections while the axial hub features may include recessed portions, the features may be annular, the features may include tabs, the features may include any other suitable shape or configuration for maintaining the position of the axial lock member 720 within the axial hub 710, or a combination there.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A catheter lock, comprising:
a hub having:
a first axial extension;
a second axial extension; and
an intersecting opening with at least one hub feature formed on an inner surface thereof;
a button having:
an outer surface mirroring the inner surface of the intersecting opening, the outer surface including at least one button feature formed thereon;
an elongated transverse opening;
a cutout section extending along the outer surface of the button; and
a hole in a top portion thereof; and
a locking article configured to extend through the first axial extension, along the cutout section, and through the hole;
wherein the button is arranged and disposed to slide within the intersecting opening of the hub; and
wherein the at least one button feature is arranged and disposed to engage one of the at least one hub features.

2. The catheter lock according to claim 1, wherein the at least one hub feature comprises a first hub feature and a second hub feature.

3. The catheter lock according to claim 2, wherein the at least one button feature comprises two sets of button features, each set including a first button feature, a second button feature, and a third button feature.

4. The catheter lock according to claim 3, wherein the first button feature of the first set engages the first hub feature and the first button feature of the second set engages the second hub feature to hold the button in a first position within the hub.

5. The catheter lock according to claim 3, wherein the second button feature of the first set engages the first hub feature and the second button feature of the second set engages the second hub feature to hold the button in a second position within the hub.

6. The catheter lock according to claim 3, wherein the third button feature of the first set engages the first hub feature and the third button feature of the second set engages the second hub feature to hold the button in a third position within the hub.

7. The catheter lock according to claim 3, wherein the third button feature of the first set of button features is positioned within the cutout section.

8. The catheter lock according to claim 1, wherein the cutout section extends from a first side of the elongated transverse opening to a second side of the elongated transverse opening.

9. The catheter lock according to claim 1, wherein the button forms a fluid-tight seal with the hub.

10. The catheter lock according to claim 1, wherein the elongated transverse opening aligns with the first axial extension and the second axial extension when the button is positioned within the hub.

11. A catheter lock, comprising:
a hub having:
a first axial extension;
a second axial extension with at least one hub feature formed on an inner surface thereof; and
a hole formed in a surface thereof;
a lock member having:
an outer surface mirroring the inner surface of the second axial extension, the outer surface including at least one lock member feature formed thereon;
an axial opening extending through the lock member; and
at least one surface element on the outer surface of the lock member, the at least one surface element configured to contact an inner surface of the hub only when the lock member is in a closed position; and
a locking article configured to extend through the first axial extension, between the outer surface of the lock member and the inner surface of the hub, and through the hole;
wherein the lock member is arranged and disposed to slide within the second axial extension of the hub;
wherein the at least one lock member feature is arranged and disposed to engage one of the at least one hub features to maintain a position of the lock member within the second axial extension; and
wherein the axial opening extending through the lock member is arranged and disposed to align with the first axial extension when the lock member is positioned within the second axial extension.

12. The catheter lock of claim 11, further comprising a grommet arranged and disposed to be positioned within the hole formed in the hub, the grommet forming a liquid tight seal with the locking article.

13. The catheter lock of claim 11, wherein the at least one lock member feature and the at least one hub feature are arranged and disposed to maintain the lock member in an open position or the closed position.

14. The catheter lock of claim 11, wherein the at least one surface element is arranged and disposed to pin the locking article between the outer surface of the lock member and the inner surface of the hub when the lock member is in the closed position.

15. A locking catheter, comprising:
the catheter lock according to claim 1; and
an elongated tube member having a proximal end and a distal end;
wherein the proximal end of the elongated tube member is coupled to the first axial extension of the catheter lock;
wherein the distal end of the elongated tube member is flexible and includes at least one opening formed therein; and
wherein the locking article is arranged and disposed to be coupled to the distal end of the elongated tube member before extending through the first axial extension and through the hole.

16. The locking catheter according to claim 15, wherein the distal end of the elongated tube member includes a first curved or looped configuration.

17. The locking catheter according to claim 16, further comprising a removable stylet, the stylet being arranged and disposed to straighten the distal end when inserted into the elongated tube member.

18. The locking catheter according to claim 15, wherein pulling a free end of the locking article extending through the hole applies tension to the locking article.

19. The locking catheter according to claim 18, wherein the tension holds a desired position of the distal end.

20. The locking catheter according to claim 19, wherein when in a closed position the catheter lock maintains the tension in the locking article to secure a position of the distal end.
